(19) United States
(12) Patent Application Publication
(10) Pub. No.: US 2014/0228954 A1
(43) Pub. Date: Aug. 14, 2014

(54) Vestibular Implant Parameter Fitting

(71) Applicant: MED-EL Elektromedizinische Geraete GmbH, Innsbruck (AT)

(72) Inventors: Roland Hessler, Innsbruck (AT); Andreas Jäger, Reith bei Seefeld (AT)

(73) Assignee: MED-EL Elektromedizinische Geraete GmbH, Innsbruck (AT)

(21) Appl. No.: 14/256,090

(22) Filed: Apr. 18, 2014

Related U.S. Application Data
(62) Division of application No. 13/606,262, filed on Sep. 7, 2012.
(60) Provisional application No. 61/532,817, filed on Sep. 9, 2011.

(51) Int. Cl. A61F 2/18; A61N 1/05; A61N 1/36

(52) U.S. Cl. CPC A61F 2/18; A61N 1/36032; A61N 1/0541; A61F 2002/183; A61F 2002/482
USPC 623/10

(57) ABSTRACT

A vestibular implant fitting system is described for fitting a vestibular implant to an implanted patient. An event processing module is configured for monitoring extra-clinical operation of the vestibular implant and collecting extra-clinical implant operation information. A signal processing fitting module is configured for determining a body response characteristic of the implanted patient to a vestibular implant stimulus signal during a response measurement period, and setting an operating characteristic of the vestibular implant based on the body response characteristic and the extra-clinical implant operation information.

This application is a divisional of U.S. patent application Ser. No. 13/606,262, filed Sep. 7, 2012, which claims priority from U.S. Provisional Patent Application 61/532,817, filed Sep. 9, 2011, which is incorporated herein by reference.

### TECHNICAL FIELD

The present invention relates to implantable stimulation systems, and more specifically to a vestibular implant system which acts as a balance organ prosthesis.

### BACKGROUND ART

A normal ear directs sounds as shown in FIG. 1 from the outer ear pinna 101 through the generally cylindrical ear canal 110 to vibrate the tympanic membrane 102 (eardrum). The tympanic membrane 102 moves the bones of the middle ear 103 (malleus, incus, and stapes) that vibrate the cochlea 104, which in turn functions as a transducer to generate electric pulses to the brain that are interpreted as sounds.

In addition to hearing, the inner ear also includes a balance sensing system which involves the vestibular labyrinth, its three interconnected and mutually orthogonal semi-circular canals: the superior canal 106, posterior canal 107, and horizontal canal 108 (as well as the otolith organs 116 in the utricle and saccule of the inner ear. The canals and otoliths of the vestibular labyrinth contain hair cells 118 in a viscous endolymph 117 to sense head orientation and head movements, thereby activating nerve fibers 119 that send an electrical balance signal via the vestibular nerve 105 to the brain.

In some people, the vestibular system is damaged or impaired. Vestibular dysfunction can cause balance problems such as unsteadiness, vertigo and unsteady vision. This can be a significant handicap in everyday life. To treat such problems, electrical stimulation of the vestibular system can help to restore the balancing function, and vestibular implants are currently under development to provide such an artificial balance signal.

FIG. 1 also shows some components of a vestibular implant (VI) system such as described in U.S. Patent Application 61/366,345 (incorporated herein by reference) that improves the patient's condition in terms of gaze and of body posture during standing and walking. An external movement signal from one or more linear and/or angular accelerometers acting as balance sensors is processed by an external processor 111 to produce a vestibular stimulation signal. An external transmitter coil 112 couples the stimulation signal through the skin to an implanted receiver coil 113. Implanted vestibular stimulator 114 than delivers the stimulation signal through an electrode lead 109 to vestibular stimulator electrodes 115 that electrically stimulate target neural tissue such as the semicircular canals 106, 107, 108, one or both otolith organs, and/or the vestibular nerve 105 or ganglion for vestibular sensation by the patient as a balance signal used to maintain balance, to walk normally, to see sharply, etc.

To maximize the benefit of the system to the patient, the implant system needs to be adjusted for each specific patient in a clinical fitting process. The fit process chooses between various possible signal processing algorithms and modifies some of the signal processing of any such algorithm. Such fittings may be done manually, automatically, or semi-automatically. Information on patient performance whilst using the implant system is needed to compare different processing algorithms and/or processing parameters with regards to any differences in the performance of the system or the experience of the patient. This information can be obtained subjectively by feedback from the patient and/or by different objective measurement methods. Presently most objective fitting measurements are performed as acute clinical tests, which are accounted for at the time of the clinical fitting session. The patient's subjective feedback can be related to acute tests performed in a clinical setting and can also include subjective feedback from the patient's recollection of past events. But a patient, especially children, may not detect or remember many potentially relevant events or be able to describe these usefully to a fitting clinician.

### SUMMARY

Embodiments of the present invention are directed to a vestibular implant fitting system and method for fitting a vestibular implant to the individual needs of an implanted patient. An event processing module is configured for monitoring extra-clinical operation of the vestibular implant and collecting extra-clinical implant operation information. A signal processing fitting module is configured for determining a body response characteristic of the implanted patient to a vestibular implant stimulus signal during a response measurement period, and setting an operating characteristic of the vestibular implant based on the body response characteristic and the extra-clinical implant operation information.

In some embodiments, the body response characteristic may include one or more of an eye movement response, body posture, body sway, and/or cardiovascular response measured during the response measurement period. The event processing module may be configured for continuously collecting the extra-clinical implant operation information during extra-clinical operation of the vestibular implant, for collecting the extra-clinical implant operation information during an event data period associated with an extra-clinical data event, or when sensing one or more of a low power condition in the vestibular implant, an operational malfunction in the vestibular implant, an unusual acceleration condition, and an abnormal patient response condition. The extra-clinical implant operation information may include sensor signal data from a vestibular implant sensor, and/or stimulation signal data related to a vestibular implant stimulation signal.

### BRIEF DESCRIPTION OF THE DRAWINGS

FIG. 1 shows various anatomical structures in a human ear having a vestibular implant system.

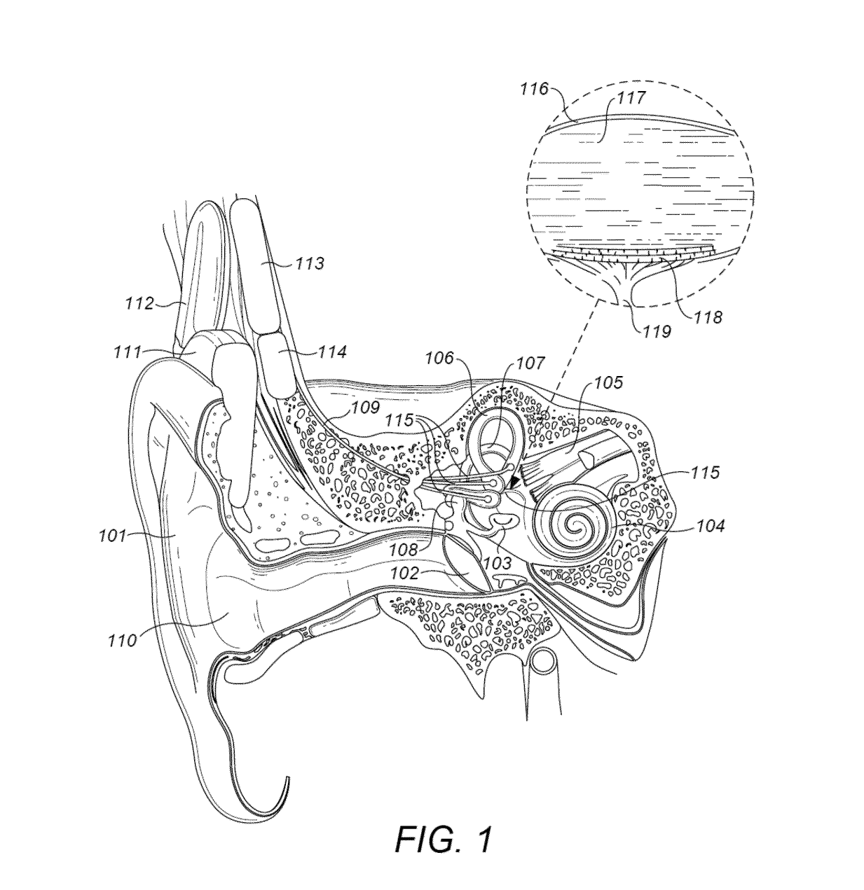

FIG. 2 shows a block diagram of a system for fitting a vestibular implant according to one specific embodiment of the present invention.

FIG. 3 shows various functional blocks in a vestibular implant fitting process according to one specific embodiment of the present invention.

FIG. 4 shows various functional blocks in a vestibular implant fitting process according to one specific embodiment based on long term event recording and analysis.

FIG. 5 shows a block diagram of a system for fitting a vestibular implant according to one specific embodiment based on long term event recording and analysis with implanted sensors.

FIG. 6 shows a block diagram of a system for fitting a vestibular implant according to one specific embodiment based on long term event recording and analysis with external sensors.

### DETAILED DESCRIPTION

Embodiments of the present invention are directed to a vestibular implant fitting system and method for automatically or semi-automatically fitting a vestibular implant to the implanted patient that adapts the stimulation signal pattern based on physiological signal.

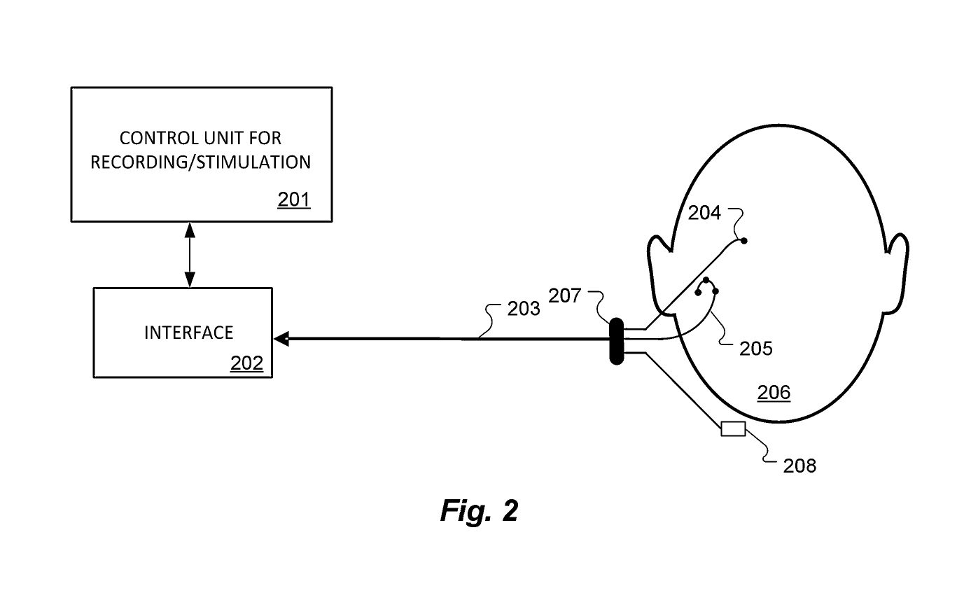

FIG. 2 shows a block diagram of a system for fitting a vestibular implant according to one specific embodiment of the present invention. Control unit 201 for recording and stimulation generates stimulation signals and analyzes response measurements. Connected to the control unit 201 is an interface box 202 that formats and distributes the input and output signals between the control unit 201 and the other system components implanted in the patient 206. For example, as shown in FIG. 2, there may be an interface lead 203 connected at one end to the interface box 202 and at the other end having an electrode plug 207 that then divides into a vestibular implant stimulation electrode 205 and a response measurement electrode 204. During a response measurement period after the stimulus signal is delivered by the vestibular implant stimulation electrode 205, the response measurement electrode 204 acts as a sensing element to measure a corresponding body response characteristic. Then based on the body response characteristic that is measured, the control unit 201 can determine how to set a related operating characteristic of the vestibular implant system.

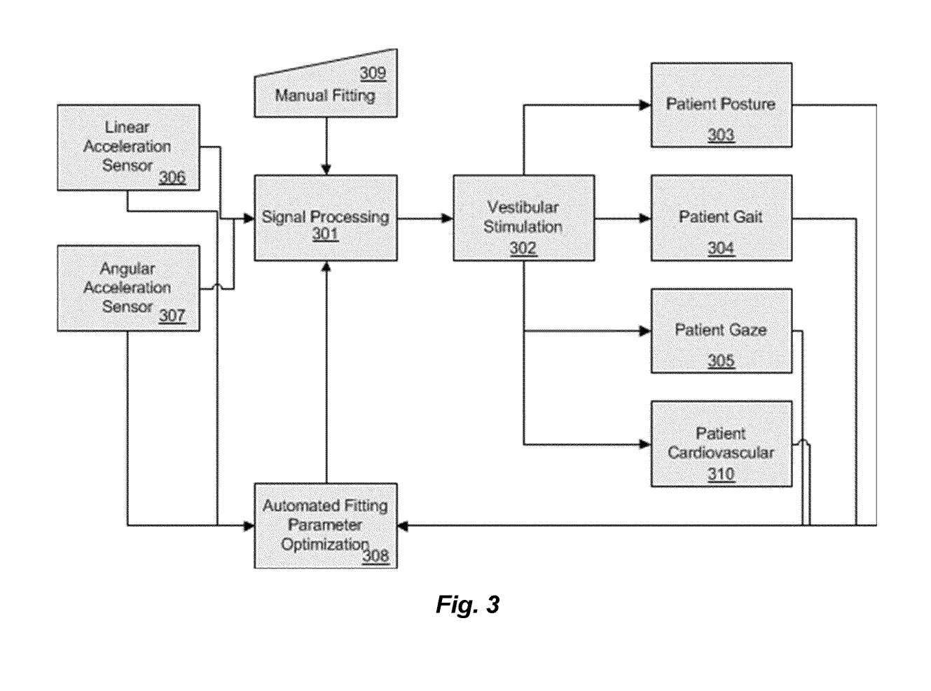

More specifically, FIG. 3 shows various functional blocks in a vestibular implant fitting process according to one specific embodiment of the present invention. Following the arrangement in FIG. 3, a signal processing block 301 receives patient motion input signals from one or more of a linear acceleration sensor 306 and/or an angular acceleration sensor 307 to produce a vestibular implant stimulus signal which is delivered to the implant electrodes by the vestibular stimulation block 302. During the response measurement period, one or more measurement sensors such as patient posture sensor 303, patient gait sensor 304, patient cardiovascular sensor 310 and/or patient gaze sensor 305 measure a body response characteristic responsive to the implant stimulus signal. An automated fitting parameter adaptation/optimization block 308 may then process the measured sensor signals, and optionally also the linear acceleration sensor 306 and/or the angular acceleration sensor 307 to adapted the vestibular implant stimulation signal produced by the signal processing block to optimize the fit of the system for that specific patient. In addition, a manual fitting block 309 may be used to fit some operating characteristics of the implant system.

For example a patient gaze sensor 305 may be implemented as an electrode array which records evoked potentials at the eye muscles, at their innervating nerves, or at the facial tissue above and/or below the eye. Or a patient gaze sensor 305 may be implemented as an optical sensor to optically measure eye movement. For example, an optical sensor may be implemented in a pair of eyeglasses as an integrated camera with an inductive link to the fitting parameter adaptation/optimization block 308 or to the signal processing block 301. For example, one eye movement that may usefully be measured includes the nystagmus, which is an eye movement that is characterized by alternating smooth pursuit of the eye in one direction and saccadic catch-up movement in the other direction to keep the image on the retina steady during head movements. If the nystagmus deviates from the healthy or normal condition the vision becomes blurry (oscillopsia).

Other system sensors such as linear acceleration sensor 306, angular acceleration sensor 307, patient posture sensor 303, patient cardiovascular sensor 310 and/or patient gait sensor 304 may be based on gyroscopes and/or acceleration sensors and can be used to detect and eventually record the stability of the patient during movements or when resting. The fitting parameter adaptation/optimization block 308 could detect from the sensor signals, for example, the amount of swaying in the gait of the patient when walking. The fitting parameter adaptation/optimization block 308 can rate and compare the sensor information on the patient performance with patient performance data from different specific stimulation patterns. Thus, the fitting parameter adaptation/optimization block 308 may change the stimulation pattern generated by the signal processing block 301 to the vestibular stimulation block 302 and compare the patient's performance with the new stimulation pattern to the previous stimulation patterns to automatically find the best fitting algorithm for the patient.

Embodiments of the present invention also provide for the extra-clinical collection of subjective and objective information beyond that of clinical fitting sessions. This provides new sources of such subjective and objective information compared to existing fitting processes that rely solely on acute clinical diagnostic measurements. Long-term recording and event analysis has been used in the past in other medical applications both for diagnostic purposes and for treatment efficacy controls, especially by means of holter devices. These medical applications include ambulant long-term recording of ECG/CRM, EEG and other physiological parameters. However, none of the known medical applications relates to the very different application of active inner ear implants or to making use of the information for the purposes of optimizing patient benefit by adjusting device signal processing settings.

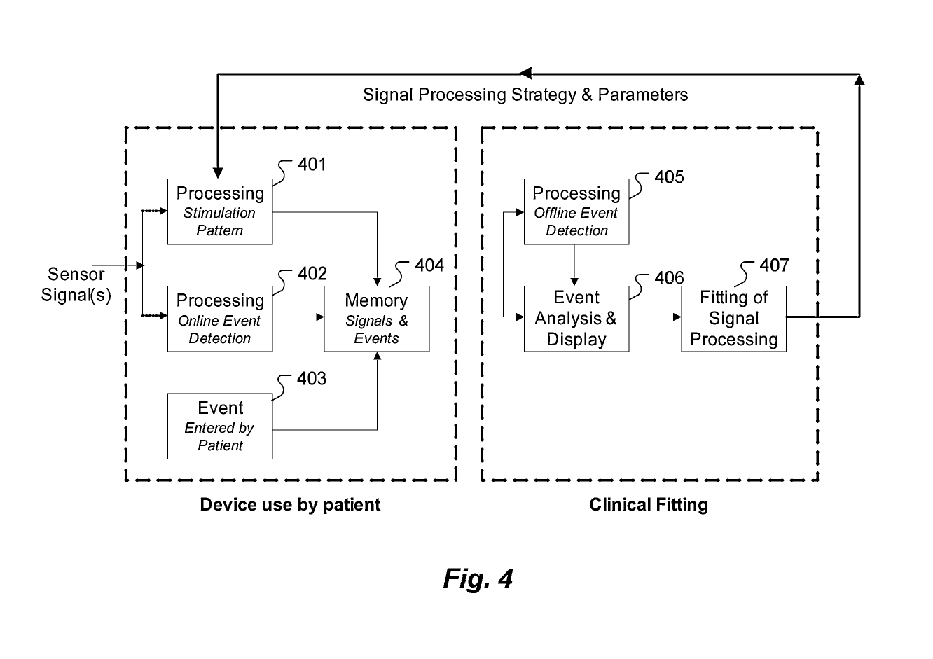

FIG. 4 shows various functional blocks in a vestibular implant fitting process according to one specific embodiment based on long term event recording and analysis. The vestibular implant system proper receives balance related sensor signals that are used for conventional stimulation pattern processing 401. The sensor signal inputs also are used for online event detection processing 402. Detected events can be utilized during the patient's regular extra-clinical use of the implant system as well as manually entered events 403. Some examples of such fitting relevant events may include without limitation:

low power status during system operation

manual event entry 403 by the patient indicating that the stimulation signal was too strong or that some undesired side-effect was experienced

a patient fall or otherwise uncommon condition (e.g., from a sensor signal online analysis by the online event detection processing 402)

automated offline analysis of the sensor signal indicates a spurious device malfunction, and/or

offline event analysis 406 (e.g., by a clinician) indicates inappropriate device use by the implant patient.

System performance signal data from the stimulation pattern processing 401 and fitting related event data from the online event detection processing 402 are recorded in implant memory 404. Examples of the data that can be recorded in the implant memory 404 include without limitation:

sensor signal raw data,

pre-processed signal data from stimulation pattern processing 401,

pre-processed signal data from online event detection processing 402,

automatically detected events and

manual event entry 403 by the patient.

Then during one or more clinical fitting sessions, the fitting data in the implant memory 404 is further processed in offline event detection processing 405. Event analysis and display 406 allows the clinician to work with the online and offline fitting data to customize the fit of the system operating parameters for the specific patient, i.e., to customize the fit of the stimulation signal processing strategy and parameters of the stimulation pattern processing 401. Event analysis and display 406 can be used to assess patient performance with the implant device in use and to compare assessed performances. Such performance comparisons for an individual patient may serve among others:

to monitor patient performance over time with one permanently used processing setting

to detect performance differences between any previous and a current processing setting

to detect performance differences between any two different processing strategies

to assess differences to standards of a patient group or a group of normal subjects

The information gained thereby can be used clinically to optimize the device's processing in regards to patient-specific needs.

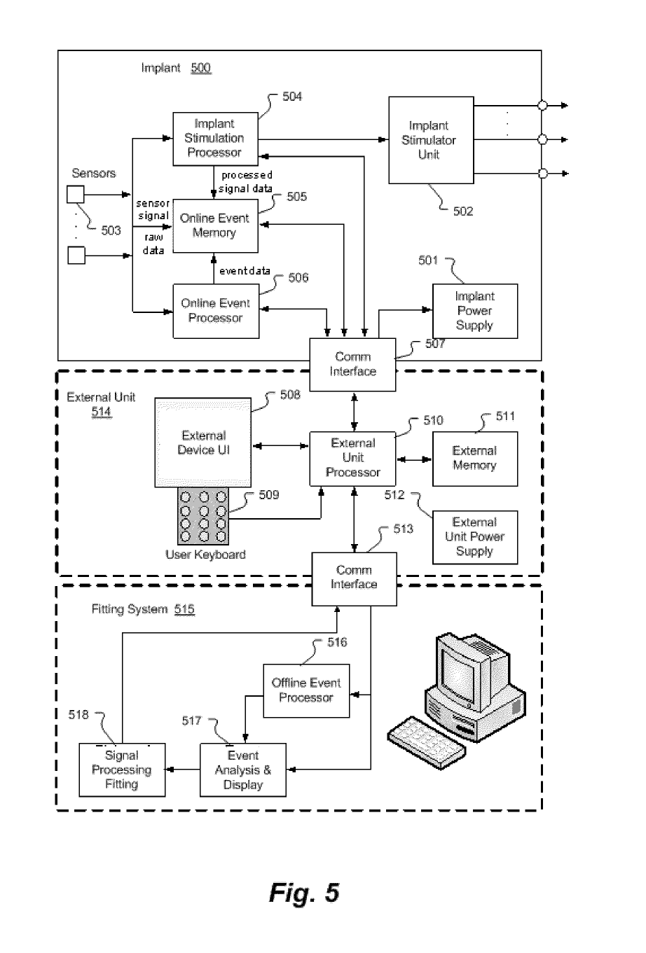

FIG. 5 shows a block diagram of an embodiment of a system for fitting a vestibular implant system with implanted sensors. The upper third of the drawing within the solid border contains the functional blocks in the implant device 500 which is powered by an implant power supply 501. An implant stimulator unit 502 produces a vestibular implant stimulus signal which is delivered to the implanted stimulation electrodes. Patient input signals are developed by one or more implanted sensors 503 which sense such patient balance related characteristics as posture, gait, gaze, and movement for an implant stimulation processor 504 that controls the implant stimulator unit 502.

An online event processor 506 monitors extra-clinical operation of the implant system and collects fitting relevant information for subsequent setting of one or more system operating characteristics. For example, the online event processor 506 can detect events of possible relevance for clinical fitting purposes based on the measured sensor signals from one or more of the implant sensors 503 and/or the signals associated with the stimulation processor 504. The data accumulated during system operation and monitoring of a fitting event can be stored in an online event memory 505 controlled by the online event processor 506.

The implant system also includes an external unit 514 that communicates with the implant device 500 via a communications interface 507, e.g., a conventional rf coil link. An external device user interface 508 with a user keyboard input 509 controls an external unit processor 510 to interact with the implant device 500 to control, program and download online event detection fitting information to an external memory 511. An power supply 512 powers the modules in the external device 514.

A clinical fitting system 515 interacts through communications interface 513 to process the event detection fitting data in the event analysis and display 517 module. The clinician works with the signal processing fitting 518 module to customize the fit of the patient device 500, specifically, the stimulation signal processing strategy and stimulation parameters and possibly the online event detection processing 506. The customized fit information and related fitting programming is passed back up through the external unit 514 to the implant device 500 to customize the operation of the implant stimulation processor 504.

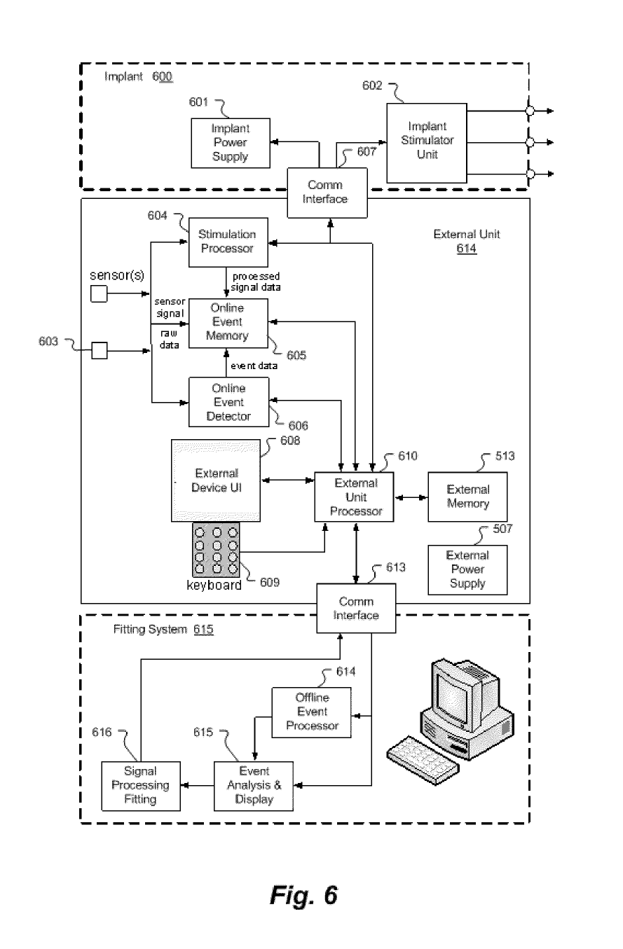

FIG. 6 shows a block diagram of a system for fitting a vestibular implant device 600 according to one specific embodiment based on long term event recording and analysis with external sensors in an external unit 614. That is, the sensors 603, stimulation processor 604, online event memory 605 and online event detector 606 are all in the external unit 614 rather than the implant device. Such an arrangement may be useful for vestibular implant systems where the implant device 600 lacks the functional structure to perform the online signal processing and/or event detection by instead providing such functionality in the external unit 614 without requiring any surgical intervention or replacement.

In various specific embodiments, event recording may be continuous, or start automatically upon detecting a trigger event and stop after some predefined time or after not detecting more events for a defined time period. Automatic event detection can be online (real-time processing) or offline. Event recording also may start and stop in response to a request by the patient (or guardian) or at preset times determined by a clinician during a fitting session. Additionally, event information may be provided/entered online by the patient (or guardian). This allows correlating recorded signals and/or automatically detected events with events that the patient experienced or perceived. Results of online event detection may be provided to the patient or others at the time of the detection, e.g. as a means of warning. Offline event detection within the clinical fitting system will utilize transfer or at least memory read-out of the related information. The event information can be used in that clinical fitting session for fitting improvement right away or for use in future fitting sessions.

An automated or semi-automated fitting of the vestibular implant to the patient may be performed initially post-surgery and/or as an optimizing adjustment of the fitting after a period of time, and/or at regular post-surgical intervals. All or part of the fitting adaptations can be performed during clinical patient visits for fitting, during remote fitting sessions, during dedicated home fitting sessions, or during regular use of the device by the patient.

The embodiments of the present invention described above form a closed-loop system, which potentially could lead to some instability of some system parameters. This should receive some attention during development and actual device use, especially since at least the human elements of such a closed loop system will likely be functioning non-linearly. Arrangements such as those described above could reduce or even eliminate the need for or at least reduce the frequency of clinical fitting sessions. This could represent a meaningful time- and cost-savings in health care. And the vestibular implant patient may benefit from a device fitting in an optimized state despite of any changes over time in the implant system or the patient, thereby increasing the benefit of the device.

Embodiments of the invention may be implemented in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components. For example, a pseudo code representation of a generic embodiment might be set forth as follows:

Process PatientFitting

body_response_characteristic (vestibular_stimulus) implant_parameter (body_response_characteristic)

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A vestibular implant fitting system for fitting a vestibular implant to an implanted patient, the system comprising:
an event processing module configured for monitoring extra-clinical operation of the vestibular implant and collecting extra-clinical implant operation information; and
a signal processing fitting module configured for:
i. determining a body response characteristic of the implanted patient to a vestibular implant stimulus signal during a response measurement period, and
ii. setting an operating characteristic of the vestibular implant based on the body response characteristic and the extra-clinical implant operation information.

2. The system according to claim 1, wherein the body response characteristic includes an eye movement response measured by a patient gaze sensor during the response measurement period.

3. The system according to claim 1, wherein the body response characteristic includes body posture of the patient measured by a patient posture sensor during the response measurement period.

4. The system according to claim 1, wherein the body response characteristic includes body sway measured by a patient gait sensor during the response measurement period.

5. The system according to claim 1, wherein the body response characteristic includes cardiovascular response measured by a patient cardiovascular sensor during the response measurement period.

6. The system according to claim 1, wherein the event processing module is configured for continuously collecting the extra-clinical implant operation information during extra- clinical operation of the vestibular implant.

7. The system according to claim 1, wherein the event processing module is configured for collecting the extra-clinical implant operation information during an event data period associated with an extra-clinical data event.

8. The system according to claim 1, wherein the event processing module is configured for collecting the extra-clinical implant operation information when sensing one or more of a low power condition in the vestibular implant, an operational malfunction in the vestibular implant, an unusual acceleration condition, and an abnormal patient response condition.

9. The system according to claim 1, wherein the extra-clinical implant operation information includes sensor signal data from a vestibular implant sensor.

10. The system according to claim 1, wherein the extra-clinical implant operation information includes stimulation signal data related to a vestibular implant stimulation signal.

11. A method for fitting a vestibular implant to an implanted patient, the method comprising:
monitoring extra-clinical operation of the vestibular implant and collecting extra-clinical implant operation information;
determining a body response characteristic of the implanted patient to a vestibular implant stimulus signal during a response measurement period; and
setting an operating characteristic of the vestibular implant based on the body response characteristic and the extra-clinical implant operation information.

12. The method according to claim 11, wherein determining a body response characteristic includes measuring an eye movement of the patient during the response measurement period.

13. The method according to claim 11, wherein determining a body response characteristic includes measuring body posture of the patient during the response measurement period.

14. The method according to claim 11, wherein determining a body response characteristic includes measuring body sway of the patient during the response measurement period.

15. The method according to claim 11, wherein determining a body response characteristics includes measuring the cardiovascular system of the patient.

16. The method according to claim 11, wherein the extra-clinical implant operation information is continuously collected during extra-clinical operation of the vestibular implant.

17. The method according to claim 11, wherein the extra-clinical implant operation information is collected during an event data period associated with an extra-clinical data event.

18. The method according to claim 11, wherein the extra-clinical implant operation information is collected based on sensing one or more of a low power condition in the vestibular implant, an operational malfunction in the vestibular implant, an unusual acceleration condition, and an abnormal patient response condition.

19. The method according to claim 11, wherein the extra-clinical implant operation information includes sensor signal data from a vestibular implant sensor.

20. The method according to claim 11, wherein the extra-clinical implant operation information includes stimulation signal data related to a vestibular implant stimulation signal.
